(19) United States
(12) Patent Application Publication
(10) Pub. No.: US 2003/0049290 A1
(43) Pub. Date: Mar. 13, 2003

(54) Deodorant composition

(76) Inventors: Brajesh Kumar Jha, Higashinada-ku (JP); Naohisa Yoshimi, Hyogo (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/216,605

(22) Filed: Aug. 9, 2002

Related U.S. Application Data
(60) Provisional application No. 60/316,780, filed on Aug. 31, 2001.

(51) Int. Cl. A61K 31/724; A61K 7/00; A61K 9/14

(52) U.S. Cl. 424/401; 514/58; 424/486

(57) ABSTRACT

The present invention relates to a composition for removing body malodor comprising a cyclodextrin, a film forming polymer and an aqueous carrier. The deodorant composition of the present invention may be in the form of a lotion that removes or eliminates body odor, provides long lasting deodorant effects, and moisturizing and antibacterial benefit to skin.

### CROSS REFERENCE TO RELATED APPLICATION

This application claims the benefit of U.S. Provisional Application No. 60/316,780, filed Aug. 31, 2001.

### FIELD OF INVENTION

The present invention relates to a deodorant composition which is effective for preventing or eliminating body malodors. In particular, the present invention relates to a deodorant composition which comprises a cyclodextrin and a film forming polymer.

### BACKGROUND OF THE INVENTION

Deodorant compositions are well known for use in controlling body malodors associated with human perspiration. These malodors develop from human perspiration primarily as the result of microbial interaction with sweat gland secretions which then produces small chain fatty acids, key body odor/foot odor organic acids. Deodorant compositions may contain deodorant actives such as antimicrobial agents to help control the microbial development of such malodors, and/or they may contain deodorizing fragrances that help to mask the sensory perception of the malodors.

Deodorant compositions are typically formulated as deodorant sticks which also contain a gellant or other structurant, and a polar alcohol solvent to help solubilize the gellant or other structurant. These deodorant formulations are typically applied topically to the underarm or other area of the skin. Although these deodorant sticks are quite popular and commonly used to control or mask malodors associated with human perspiration, many of these alcohol-containing deodorant sticks may also be harsh to the skin and may cause excessive skin irritation, burning and itching after topical application. These irritation, burning and itching which may be caused by such deodorant sticks make them less suitable for application to other areas of the body which are more sensitive than the underarm.

Other attempts at controlling body malodors include the use of odor absorbers such as activated charcoal and zeolites. Deodorant compositions which contain these malodor absorbing agents are typically formulated as aqueous lotions, aqueous roll-ons, and aqueous soft deodorant gels which comprise the odor absorber, and an aqueous liquid carrier. These activated charcoal and zeolite odor absorbing agents, however, may be ineffective when wet and are known to be less efficient at absorbing odors when they are included in aqueous systems, especially when the aqueous compositions are applied to the skin and the activated charcoal or zeolite comes in contact with human body fluids such as sweat.

Thus, there exists a need for anti-odorant compositions which cause less irritation or uncomfortable sensation when applied to the skin, and which has long lasting deodorant benefit after the compositions have been topically applied to the skin.

### SUMMARY OF THE INVENTION

The present invention is directed to a deodorant composition for removing body odor comprising a cyclodextrin, a film forming polymer and an aqueous carrier.

The present invention is also directed to a wet wipe comprising one or more layers of a water-insoluble substrate and a deodorant composition for removing body odor, the deodorant composition comprising from about 1% to about 10% by weight of the composition of a cyclodextrin, from about 0.10% to about 1.0% by weight of the composition of a film forming polymer, and an aqueous carrier.

These and other features, aspects, and advantages of the present invention will become evident to those skilled in the art from a reading of the present disclosure.

### DETAILED DESCRIPTION OF THE INVENTION

It has been surprisingly found that a deodorant composition may be formulated in a form of lotion to contain a cyclodextrin and a film forming polymer. A cyclodextrin in combination with a film forming polymer provides synergistic deodorant effects on body odorant. The deodorant composition comprising a cyclodextrin and a film forming polymer provides improved body malodor control without resulting in skin irritation and provide for long lasting deodorant benefit after the composition has been topically applied to the skin.

The term “body odor” or “body malodor”, as used herein, means odors which are generated as a result of the natural functioning of a human or mammalian body. Such odors include, but are not limited to, odors produced by microorganisms of the human or mammalian skin (i.e., bacterial decomposition of skin secretions) or urine, and mixtures thereof. Such odors are mainly organic molecules, which have different structures and functional groups, such as amines, acids, alcohols, aldehydes, ketones, phenolics, and polycyclics including aromatics, polyaromatics. These molecules may also be made up of sulphers containing functional groups, such as thiol, mercaptan, sulfide and/or disulfide groups.

The term “deodorant effects” means an activity of substantially reducing or eliminating unpleasant odors and more particularly body odor.

The deodorant compositions of the present invention may comprise, consist of, or consist essentially of the essential elements and limitations of the present invention described herein, as well as any of the additional or optional ingredients, components, or limitations described herein.

All percentages, parts and ratios are by weight of the total composition, unless otherwise specified. All such weights as they pertain to listed ingredients are based on the specific ingredient level and, therefore, do not include solvents, carriers, by-products, filler or other minor ingredients that may be included in commercially available materials, unless otherwise specified.

Cyclodextrins

The deodorant compositions of the present invention comprise cyclodextrin. Cyclodextrins are safe and mild to the skin and reduce the body odor by encapsulating the small chain fatty acids (SCFAs) which are produced by bacterial decomposition of skin secretion. Examples of such small chain fatty acids include C1-C7 organic acid, such as 3-methyl-2-hexenoic acid and isovaleric acid. Cyclodextrins may absorb or encapsulate those SCFAs.

The cyclodextrin may be used individually or as a mixture of cyclodextrins, provided that the cyclodextrin is capable of preventing or eliminating malodors associated with perspiration.

The cyclodextrins for use in the deodorant compositions of the present invention include those cyclic polysaccharide compounds containing from 6 to 12 glucose units. The specific coupling and conformation of the glucose units enable the cyclodextrin to form a rigid, conical molecular structure that has a hollow interior or cavity.

The cyclodextrins suitable for use herein are preferably included in the deodorant composition of the present invention as an uncomplexed cyclodextrin. The term “uncomplexed cyclodextrin” as used herein means that the cavities within the cyclodextrin are essentially unfilled while the cyclodextrin is added into the aqueous carrier component of the compositions of the present invention.

As will be apparent to those skilled in the art, the preferred uncomplexed cyclodextrin may form inclusion complexes with the other essential and/or optional components described herein. Therefore, it is preferred that at least an effective amount of the cyclodextrin be included in the deodorant composition herein as an uncomplexed cyclodextrin, and should remain as an uncomplexed cyclodextrin until the composition has been applied to the skin. In this context, the term “effective amount” means an amount of the cyclodextrin that is in its uncomplexed form when the cyclodextrin is added into the aqueous carrier component described herein, and that remains uncomplexed until the cyclodextrin comes in contact with human body fluid such as sweat after the compositions have been topically applied to the skin.

The concentration of the cyclodextrin may vary with each selected deodorant formulation. Generally, the deodorant compositions of the present invention comprise the cyclodextrin at concentrations ranging from about 1.0% to about 10.0%, preferably from about 2.0% to about 9.0%, more preferably from about 2.5% to about 8.0%, most preferably from about 3.0% to about 7.0%, by weight of the composition.

Cyclodextrins for use herein include any of the known cyclodextrins such as unsubstituted cyclodextrins containing from 6 to 12 glucose units. Specific nonlimiting examples of such cyclodextrins include alpha-cyclodextrin, beta-cyclodextrin, gamma-cyclodextrin, delta-cyclodextrin, epsilon-cyclodextrin, zeta-cyclodextrin, nu-cyclodextrin, and mixtures thereof, and/or their derivatives, and/or mixtures thereof.

Suitable cyclodextrin derivatives include those cyclodextrin compounds of different degrees of substitution, specific examples of which include methyl-alpha-cyclodextrin, methyl-beta-cyclodextrin, hydroxyethyl-beta-cyclodextrin, hydroxypropyl-alpha-cyclodextrin, hydroxypropyl-beta-cyclodextrin, cyclodextrin glycerol ethers, maltose-bonded cyclodextrins, cationic cyclodextrins, quaternary ammonium cyclodextrins, anionic cyclodextrins such as carboxymethyl cyclodextrins, cyclodextrin sulfobutylethers, cyclodextrin sulfates, cyclodextrin succinylates, amphoteric cyclodextrins such as carboxymethyl/quaternary ammonium cyclodextrins, mono-3-6-anhydrocyclodextrins, and combinations thereof. Other examples of suitable cyclodextrin derivatives are disclosed in “Optimal Performances with Minimal Chemical Modification of Cyclodextrins”, F. Diedaini-Pilard and B. Perly, The 7th International Cyclodextrin Symposium Abstracts, April 1994, p.49; U.S. Pat. No. 3,426,011, issued to Parmerter et al. on Feb. 4, 1969; U.S. Pat. Nos. 3,453,257, 3,453,258, 3,453,259, and 3,453,260, all issued to Parmerter et al. on Jul. 1, 1969; U.S. Pat. No. 3,459,731, issued to Gramera et al. on Aug. 5, 1969; U.S. Pat. No. 3,553,191, issued to Parmerter et al. on Jan. 5, 1971; U.S. Pat. No. 3,565,887, issued to Parmerter et al. on Feb. 23, 1971; U.S. Pat. No. 4,535,152, issued to Szejtli et al. on Aug. 13, 1985; U.S. Pat. No. 4,616,008, issued to Hirai et al. on Oct. 7, 1986; U.S. Pat. No. 4,638,058, issued to Brandt et al. on Jan. 20, 1987; U.S. Pat. No. 4,746,734, issued to Tsuchiyama et al. on May 24, 1988; and U.S. Pat. No. 4,678,598, issued to Ogino et al. on Jul. 7, 1987; all of which disclosures are incorporated by reference herein.

Other suitable cyclodextrin materials for use herein include those individual cyclodextrins linked together, e.g., using multifunctional agents, to form oligomers, or other polymers. Nonlimiting examples of such materials include cyclodextrin polymers that are formed by crosslinking a cyclodextrin monomer with an aromatic, aliphatic, or cycloaliphatic polyfunctional crosslinking agent. Suitable cyclodextrin monomer materials include, but are not limited to, alpha-cyclodextrin, beta-cyclodextrin, gamma-cyclodextrin, delta-cyclodextrin, epsilon-cyclodextrin, zeta-cyclodextrin, nu-cyclodextrin, substituted alpha-cyclodextrin, substituted beta-cyclodextrin, and substituted gamma-cyclodextrin. Branched cyclodextrin monomer materials are also suitable for use herein. Specific examples of suitable polyfunctional crosslinking agents include, but are not limited to, diisocyanates, polyisocyanates, dihalohydrocarbons, and dihaloacetylhydrocarbons. Other suitable polyfunctional crosslinking agents may include asymmetric crosslinking agents containing different linking functionalities such as isocyanate, halo, or haloacetyl, an example of which include 4-isocyanatobenzoyl chloride. Specific examples of cyclodextrin polymers that are suitable for use herein include, but are not limited to, beta-cyclodextrin crosslinked by epichlorohydrin and ethyleneglycolbis (epoxypropyl ether); and alpha-, beta-, or gamma-cyclodextrin crosslinked by a polyisocyanate or dihalohydrocarbon polyfunctional crosslinking agent. Other polymeric forms are also suitable for use herein, such as carboxylic acid containing polymer-cyclodextrin conjugates which may be prepared by conjugating a suitable carboxylic acid containing polymer to a cyclodextrin monomer using any method well known in the art for preparing cyclodextrin polymers.

Preferred cyclodextrins suitable for use in the deodorant composition of the present invention include alpha-cyclodextrin, beta-cyclodextrin, methyl-alpha-cyclodextrin, methyl-beta-cyclodextrin, hydroxypropyl-alpha-cyclodextrin, hydroxypropyl-beta-cyclodextrin, or mixtures thereof. Beta-cyclodextrin is more preferred.

Alpha-cyclodextrin, beta-cyclodextrin, gamma-cyclodextrin and/or their derivatives may be obtained from, among others, Cerestar USA, Inc., located in Hammond, Ind.; Wacker Chemicals (USA), Inc., located in New Canaan, Conn.; Aldrich Chemical Company located in Milwaukee, Wis.; and Chinoin Pharmaceutical Works located in Budapest, Hungary.

It is also preferable to use a mixture of cyclodextrins. Such mixtures absorb perspiration malodors more broadly by complexing with odoriferous molecules that may vary widely in size. Mixtures of cyclodextrin may conveniently be obtained by using intermediate products from known processes for the preparation of cyclodextrins, examples of which include those processes described in U.S. Pat. No. 3,425,910 issued to Armbruster et al. on Nov. 29, 1983; and U.S. Pat. No. 4,738,923, issued to Ammeraal on Apr. 19, 1988; both descriptions of which are incorporated by reference herein. Preferably, at least a major portion of the cyclodextrin mixtures is alpha-cyclodextrin, beta-cyclodextrin, and/or gamma-cyclodextrin, more preferably beta-cyclodextrin. Some commercial examples of cyclodextrin mixtures are available from Ensuiko Sugar Refining Company located in Yokohama, Japan.

Film Forming Polymer

The film forming polymers for use in the deodorant composition of the present invention is a component that dries completely at room conditions to form a continuous film that helps deposit other components on the skin.

The film forming polymers inhibit perspiration and hence the growth of bacteria activity which breaks down elements contained in sweat or urine into SCFAs. It has been surprisingly found that the combination of the film forming polymers with cyclodextrins provides long lasting deodorant benefit. Further, the presence of film forming polymers adds conditioning and moisturizing benefit to the skin.

The film forming polymers of the present invention are water-soluble so that they may be solubilized in an aqueous carrier. The film forming polymer suitable for the present invention has a number average molecular weight of at least 100,000 or more. Preferred film forming polymers have number average molecular weight of about 4×10⁶ or more. The film forming polymers having number average molecular weight of at least 100,000 or more are believed to be deposited on the skin longer than polymers having number average molecular weight of less than 100,000. Film forming polymers having number average molecular weight of less than 100,000 may penetrate into the skin because of its size and thus are not preferred.

Nonlimiting examples of suitable film forming polymers include, acrylamide/ammonium acrylate copolymer, acrylamides copolymer, acrylamides/DMAPA acrylates/methoxy PEG methacrylate copolymer, acrylamide/sodium acrylate copolymer, acrylamidopropyltrimonium chloride/acrylates copolymer, acrylamidopropyltrimonium chloride/acrylamide copolymer, acrylates/acetoacetoxyethyl methacrylate copolymer, acrylates/acrylamide copolymer, acrylates/ammonium methacrylate copolymer, acrylates copolymer, acrylates/diacetoneacrylamide copolymer, acrylates/dimethicone copolymer, acrylates/dimethylaminoethyl methacrylate copolymer, acrylates/hydroxyesters acrylates copolymer, acrylates/octylacrylamide copolymer, acrylates/octyl acrylate copolymer, acrylates/PVP copolymer, acrylates/VA copolymer, acrylates/VA crosspolymer, acrylic acid/acrylonitrogens copolymer, adipic acid/CHDM/MA neopentyl glycol/trimellitic anhydride copolymer, adipic acid/diethylene glycol/glycerin cross polymer, adipic acid/diethylenetriamine copolymer, adipic acid/dimetylaminohydroxypropyl diethylenetriamine copolymer, adipic acid/epoxypropyl diethylenetriamine copolymer, adipic acid/fumaric acid/phthalic acid/ tricyclodecane dimethanol copolymer, adipic acid/isophthalic acid/neopentyl glycol/trimethylolpropane copolymer, adipic acid/neopentyl glycol/trimellitic anhydride copolymer, albumen, allyl stearate/VA copolymer, aminoethylacrylate phosphate/acrylates copolymer, ammonium acrylates/acrylonitrogens copolymer, ammonium acrylates copolymer, ammonium alginate, ammonium polyacrylate, ammonium styrene/acrylates copolymer, ammonium VA/acrylates copolymer, AMP-acrylates copolymer, AMP-acrylates/diacetoneacrylamide copolymer, AMP-acrylates/dimethylaminoethylmethacrylate copolymer, AMPD-acrylates/diacetoneacrylamide copolymer, Balsam Canada (Abies Balsamea), Balsam Copaiba (Copaifera Officinalis), Balsam Oregon (Pseudotsuga Menziesi), Balsam Peru (Myroxylon Pereirae), Balsam Tolu (Myroxylon Balsamum), benzoic acid/phthalic anhydride/pentaerythritol/neopentyl glycol/palmitic acid copolymer, benzoin gum, butadiene/acrylonitrile copolymer, butoxy chitosan, butyl acrylate/hydroxyethyl methacrylate copolymer, butyl acrylate/styrene copolymer, butylated polyoxymethylene urea, butylated PVP, butyl benzoic acid/phthalic anhydride/trimethylolethane copolymer, butyl ester of ethylene/MA copolymer, butyl ester of PVM/MA copolymer, calcium carboxymethyl cellulose, calcium carrageenan, calcium/sodium PVM/MA copolymer, C1-5 alkyl galactomannan, carboxybutyl chitosan, carboxymethyl chitosan, carboxymethyl chitosan succinamide, carboxymethyl dextran, carboxymethyl hydroxyethylcellulose, caster oil/IPDI copolymer, cellulose acetate, cellulose acetate butyrate, cellulose acetate propionate, cellulose acetate propionate carboxylate, cellulose gum, cetearyl dimethicone/vinyl dimethicone crosspolymer, chitosan, chitosan adipate, chitosan ascorbate, chitosan formate, chitosan glycolate, chitosan lactate, chitosan PCA, chitosan salicylate, chitosan succinamide, collodion, copal, corn starch/acrylamide/sodium acrylate copolymer, DEA-styrene/acrylates/DVB copolymer, diethylene glycolamine/epichlorohydrin piperazine copolymer, diglycol/CHDM/isophthalates/SIP copolymer, diglycol/isophthalates/SIP copolymer, dihydroxyethyl tallowamine/IPDI copolymer, dilinoleyl alcohol/IPDI copolymer, dimethicone/sodium PG-propyldimethicone thiosulfate copolymer, dimethiconol/IPDI copolymer, DMAPA acrylates/acrylic acid/acrylonitrogens copolymer, dodecanedioic acid/cetearyl alcohol/glycol copolymer, ethylcellulose, ethylene/acrylic acid copolymer, ethylene/acrylic acid/VA copolymer, ethylene/calcium acrylate copolymer, ethylene/MA copolymer, ethylene/magnesium acrylate copolymer, ethylene/methacrylate copolymer, ethylene/propylene copolymer, ethylene/sodium acrylate copolymer, ethylene/VA copolymer, ethylene/zinc acrylate copolymer, ethyl ester of PVM/MA copolymer, flexible collodion, galactoarabinan, glycereth-7 hydroxystearate/IPDI copolymer, glyceryl polyacrylate, glyceryl polymethacrylate, Gutta Percha, hydrogenated rosin, hydrogenated styrene/butadiene copolymer, hydrolyzed wheat protein/dimethicone copolyol phosphate copolymer, hydroxybutyl methylcellulose, hydroxyethylcellulose, hydroxyethyl chitosan, hydroxyethyl ethylcellulose, hydroxypropylcellulose, hydroxypropyl chitosan, hydroxypropyl guar, hydroxypropyl methylcellulose, isobutylene/MA copolymer, isobutylene/sodium maleate copolymer, isopropyl ester of PVM/MA copolymer, lauryl acrylate/VA copolymer, lauryl methacrylate/glycol dimethacrylate copolymer, maltodextrin, mannan, methacryloyl ethyl betaine/acrylates copolymer, methyl methacrylate crosspolymer, nitrocellulose, nylon-12/6/66 copolymer, octadecene/MA copolymer, octylacrylamide/acrylates/butylaminoethyl methacrylate copolymer, phthalic anhydride/adipic acid/caster oil/neopentyl glycol/PEG-3/trimethylolpropane copolymer, phthalic anhydoride/benzoic acid/trymethylolpropane copolymer, phthalic anhydride/butyl benzoic acid/propylene glycol copolymer, phthalic anhydride/glycerin/glycidyl decanoate copolymer, phthalic anhydride/trimellitic anhydride/glycols copolymer, piperylene/butene/pentene copolymer, polyacrylamide, polyacrylamidomethylpropane sulfonic acid, polyacrylic acid, polybeta-alanine, polybeta-alanine/glutaric acid crosspolymer, polybutyl acrylate, polybutylene terephthalate, polychlorotrifluoroethylene, polydiethyleneglycol adipate/IPDI copolymer, polydimethylaminoethyl methacrylate, polyethylacrylate, polyethylene, polyethylene terephthalate, polyethylglutamate, polyethylmethacrylate, polyglucuronic acid, polyisobutene, polylysine, polymethacrylamidopropyltrimonium methosulfate, polymethacrylic acid, polymethyl acrylate, polymethylglutamate, polymethyl methacrylate, polyoxyisobutylene/methylene urea copolymer, polyoxymethylene melamine, polypentene, polyperfluoroperhydrophenanthrene, poly-p-phenylene terephthalamide, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-43, polyquaternium-44, polyquaternium-45, polyquaternium-46, polyquaternium-47, polysilicone-6, polysilicone-8, polysilicone-11, polystyrene, polyvinyl acetate, polyvinyl alcohol, polyvinyl butyral, polyvinylcaprolactam, polyvinyl chloride, polyvinyl imidazolinium acetate, polyvinyl laurate, polyvinyl methyl ether, polyvinyl stearyl ether, potassium carbomer, potassium carrageenan, PPG-12/SMDI copolymer, PPG-7/succinic acid copolymer, PPG-26/TDI copolymer, PVM/MA copolymer, PVM/MA decadiene crosspolymer, PVP, PVP/dimethiconylacrylate/polycarbamyl/polyglycol ester, PVP/dimethylaminoethylmethacrylate copolymer, PVP/dimethylaminoethylmethacrylate/polycarbamyl polyglycol ester, PVP/eicosene copolymer, PVP/hexadecene copolymer, PVP/polycarbamyl polyglycol ester, PVP/VA copolymer, PVP/VA/itaconic acid copolymer, PVP/VA/vinyl propionate copolymer, quaternium-22, rosin, rubber latex, serum albumin, shellac, sodium acrylates/acrolein copolymer, sodium acrylates copolymer, sodium acrylate/vinyl alcohol copolymer, sodium carbomer, sodium carboxymethyl chitin, sodium carboxymethyl starch, sodium carrageenan, sodium C4-12 olefin/maleic acid copolymer, sodium DVB/acrylates copolymer, sodium isooctylene/MA copolymer, sodium MA/diisobutylene copolymer, sodium polyacrylate, sodium polymethacrylate, sodium polystyrene sulfonate, sodium PVM/MA decadiene crosspolymer, sodium styrene/acrylates copolymer, sodium tauride acrylates/acrylic acid/acrylonitrogens copolymer, starch/acrylates/acrylamide copolymer, starch diethylaminoethyl ether, stearamidopropyl dimethicone, steareth-10 allyl ether/acrylates copolymer, stearylvinyl ether/MA copolymer, styrene/acrylates/acrylonitrile copolymer, styrene/acrylates/ammonium methacrylate copolymer, styrene/acrylates copolymer, styrene/allyl benzoate copolymer, styrene/DVB copolymer, styrene/isoprene copolymer, styrene/MA copolymer, styrene/methylstyrene/indene copolymer, styrene/PVP copolymer, styrene/VA copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose benzoate/sucrose acetate isobutyrate copolymer, TEA-acrylates/acrylonitrogens copolymer, terephthalic acid/isophthalic acid/sodium isophthalic acid sulfonate/glycol copolymer, tosylamide/epoxy resin, tosylamide/formaldehyde resin, tragacanth (Astragalus Gummifer) gum, tricontanyl PVP, trimethylpentanediol/isophthalic acid/trimellitic anhydride copolymer, tromethamine acrylates/acrylonitrogens copolymer, VA/butyl maleate/isobornyl acrylate copolymer, VA/crotonates copolymer, VA/crotonates/methacryloxybenzophenone-1 copolymer, VA/crotonates/vinyl neodecanoate copolymer, VA/crotonates/vinyl propionate copolymer, VA/crotonic acid/PEG-20M copolymer, VA/DBM copolymer, VA/isobutyl maleate/vinyl neodecanoate copolymer, VA/vinyl butyl benzoate/crotonates copolymer, vinyl acetate, vinyl caprolactam/PVP/dimethylaminoethyl methacrylate copolymer, yeast betaglucan, yeast polysaccharides and zein.

Preferred film forming polymers are water soluble film forming polymers, such as acrylates/acetoacetoxyethyl methacrylate copolymer, acrylates/acrylamide copolymer, acrylates/ammonium methacrylate copolymer, acrylates copolymer, acrylates/diacetoneacrylamide copolymer, acrylates/dimethicone copolymer, acrylates/dimethylaminoethyl methacrylate copolymer, acrylates/hydroxyesters acrylates copolymer, acrylates/octylacrylamide copolymer, acrylates/octyl acrylate copolymer, acrylates/PVP copolymer, acrylates/VA copolymer, acrylates/VA crosspolymer, ethyl ester of PVM/MA copolymer, butyl ester of PVM/MA copolymer, isopropyl ester of PVM/MA copolymer, polyacrylamide, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-43, polyquaternium-44, polyquaternium-45, polyquaternium-46, polyquaternium-47, polyvinyl alcohol, PVP, PVP/dimethiconylacrylate/polycarbamyl/polyglycol ester, PVP/dimethylaminoethylmethacrylate copolymer, PVP/dimethylaminoethylmethacrylate/polycarbamyl polyglycol ester, PVP/eicosene copolymer, PVP/hexadecene copolymer, PVP/polycarbamyl polyglycol ester, PVP/VA copolymer, PVP/VA/itaconic acid copolymer, PVP/VA/vinyl propionate copolymer, VA/crotonates/vinyl neodecanoate copolymer.

Particularly, water soluble polymers having cationic functionalities are suitable for the deodorant composition of the present invention. Since human skin typically has a negative charge (and since opposite charges attract), the polymer having cationic functionalities tend to adhere to the anionic charges of the skin, and the polymer will deposit for longer period. The water soluble polymers having cationic functionalities are, for example, those containing amino functionalities, and amido functionalities. The preferred water soluble polymers having cationic functionalities include, but are not limited to, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-43, polyquaternium-44, polyquaternium-45, polyquaternium-46, polyquaternium-47.

Water soluble polymers containing both cationic and anionic functionalities may also be suitable. An example of water soluble polymers containing both cationic and anionic functionalities is a copolymer of dimethyldially ammonium chloride and acrylic acid, commercially available under the trade name Merquat © from Calgon. Polyquaternium-39 is preferred and may be commercially available with trade names MERQUAT PLUS 3330 and MERQUAT PLUS 3331.

Concentrations of the film forming polymer in the deodorant compositions ranges from about 0.1% to about 0.5%, preferably from about 0.2% to about 0.4%, by weight of the deodorant composition.

Aqueous Carrier

The compositions of the present invention comprise an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, the aqueous carrier is substantially water. Deionized water is preferably used. Water from natural sources including mineral cations can also be used, depending on the desired characteristic of the product.

The pH of the present composition is preferably from about 4 to about 8, more preferably from about 5 to about 7.0. Buffers and other pH adjusting agents can be included to achieve the desirable pH.

The aqueous carrier will typically comprise from about 60% to about 98% of the composition of the present invention. Preferably the composition of the present invention comprises the aqueous carrier from about 80% to about 95% by weight of the composition.

Optional Components

In addition to the essential components described hereinbefore, the deodorant compositions of the present invention may further comprise one or more optional components which may modify the physical or chemical characteristics of the compositions or serve as additional “active” components when deposited on the skin, provided that the optional components are physically and chemically compatible with the essential components described herein, or do not otherwise unduly impair product stability, aesthetics, or performance.

Preferred optional components are moisturizers, skin protectants and/or emollients. These components enhance the skin feel characteristics and/or skin care benefits of the present invention.

Moisturizers

Moisturizer can aid in adding moisture to the skin and increase the water content of the top layers of skin, and may be included in the present invention. Preferred moisturizers useful in the present invention are water soluble humectants. The composition of the present invention comprises from about 0.1% to about 20%, preferably from about 2.0% to about 15% of a water soluble humectant. Water soluble humectants useful herein include polyhydric alcohols such as glycerin, sorbitol, propylene glycol, butylene glycol, hexylene glycol, ethoxylated glucose, 1,2-hexane diol, 1,2-pentane diol, hexanetriol, dipropylene glycol, erythritol, trehalose, diglycerin, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, and mixtures thereof.

Water soluble humectants useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

Commercially available humectants herein include: glycerin with trade names STAR and SUPEROL available from The Procter & Gamble Company, CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; sorbitol with tradenames LIPONIC series available from Lipo, SORBO, ALEX, A-625, and A-641 available from ICI, and UNISWEET 70, UNISWEET CONC available from UPI; dipropylene glycol with the same tradename available from BASF; diglycerin with tradename DIGLYCEROL available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate available from Chisso Corp, the same with tradenames ACTIMOIST available from Active Organics, AVIAN SODIUM HYALURONATE series available from Intergen, HYALURONIC ACID Na available from Ichimaru Pharcos; sodium adenosin phosphate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; polyethylene glycols with the tradename CARBOWAX series available from Union Carbide, and a mixture of glyceryl polymethacrylate, propylene glycol and PVM/MA copolymer with tradename Lubrajel Oil available from Guardian Lab.

Skin Protectants

Skin protectants may prevent or reduce chafing, skin irritation and/or skin friction that may occur between skin-to-skin contact sites. The preferred skin protectants useful in the present invention include, but are not limited to, cod liver oil, cocoa butter, dimethicone, shark liver oil, petrolatum, white petrolatum, and jojoba oil. More preferred are dimethicone and jojoba oil. The composition of the present invention comprises about 0.1% to about 10%, preferably from about 0.2% to about 5.0% of the skin protectants.

Emollients

Emollients which help to maintain the soft and smooth, and pliable appearance of skin are useful in the present invention. Emollients function by their ability to remain on the skin surface or in the stratum corneum to act as lubricants, to reduce flaking, and to improve the skin appearance. Emollients useful herein include, but are not limited to, triglycerides such as vegetable oils (e.g., avocado oil, sunflower seed oil) or mineral oils; esters such as oleyl oleate, isopropyl palmitate, or isopropyl myristate; hydrocarbons such as liquid petrolatum comprising C16-C32 linear or branched hydrocarbon, liquid polyisobutylene, squalane, pristine paraffin; fatty acid higher alcohol such as oleyl alcohol. Lanolin and its derivatives, phospholipids are also useful for the composition of the present invention. The composition of the present invention comprises from about 0.01% to about 5%, preferably from about 0.05% to about 1% of an emollient.

Solubilizing Aid

The composition of the present invention may optionally but preferably contain a solubilizing aid to solubilize any components that are not readily soluble in the composition to form a low viscosity solution. A suitable solubilizing aid is surfactant, preferably no-foaming or low-foaming surfactant. Suitable surfactants are non-ionic surfactants, cationic surfactants, amphoteric surfactants and mixtures thereof. Suitable surfactants may be emulsifiers. Preferred surfactants include, but are not limited to, sorbitan derivatives such as polysorbate 20 available under tradename Tween 20 from ICI Surfactant; polyethylene glycol-polypropylene glycol block copolymers, such as Pluronic® and Pluronic R® surfactants from BASF; Tetronic® and Tetronic R® surfactants from BASF, ethoxylated branched aliphatic diols such as Surfynol® surfactants from Air Products; ethoxylated alkyl phenols, such as Igepal® surfactants from Rhone-Poulenc; ethoxylated aliphatic alcohols and carboxylic acids; polyethylene glycol diesters of fatty acids such as Nikkol® MIL-10, fatty acid esters of ethoxylated sorbitans, and mixtures thereof.

Total solubilizing agent level used in the present compositions is from 0.01% to about 20.0%, preferably from about 0.05% to about 10.0% by weight of the composition.

Antimicrobial Agents

The composition of the present invention may optionally but preferably contain solubilized, mild, antimicrobial agents which kills microorganisms or prevents or inhibits their growth and reproduction. The inclusion of the antimicrobial agents aids in increasing storage stability of the present invention. Preferred preservatives include, but are not limited to, ethanol, methyl paraben, sodium hydroxymethylglycinate, cyclic organic nitrogen compounds including imidazolidinedione compounds and polymethoxybicyclic oxazolidine; phenyl and phenoxy compounds including benzyl alcohol, 2-phenoxyethanol and hexamidine isethyonate, low molecular weight aldehyde including formaldehyde and glutaraldehyde; halogenated compounds including chlorhexidine, chlorobutanol and dibromopropamidine; and mixture thereof.

The antimicrobial agents may also be useful, for example, in controlling acne. Preferred antimicrobial agents useful in the present invention are benzoyl peroxide, erythromycin, tetracycline, clindamycin, azelaic acid, sulfur resorcinol, phenoxyethanol, and Irgasan™ DP 300 (Ciba Geigy Corp., U.S.A.).

The preferred level of antimicrobial agent is from about 0.001% to about 10%, more preferably from about 0.01% to about 5.0% by weight of the composition.

Active Components

The compositions herein may further contain other active components, which are suitable for rendering the compositions more cosmetically or aesthetically acceptable or to provide them with additional usage benefits. Examples of those active agents include, but are not limited to, skin treatment agents, plant extracts, UV protecting agents, whitening agents, anti-oxidants and radical scavengers, anti-inflammatory agents, and antimicrobial agents.

Skin treatment agents help repair and replenish the natural moisture barrier function of the epidermis, thereby providing skin benefits. Skin treatment agents useful herein are niacinamide, nicotinic acid and its esters, nicotinyl alcohol, panthenol, panthenyl ethyl ether, n-acetyl cysteine, n-acetyl-L-serine, phosphodiesterase inhibitors, trimethyl glycine, urea, gelatin, soluble collagen, royal jelly, tocopheryl nicotinate, and vitamin D3 and analogues or derivatives, and mixtures thereof.

Plant extracts useful herein are those which have an astringent type of effect for reducing the size of pores, or inhibition effect of 5-α-reductase, and are compatible with the aqueous form of the present composition, and preferably do not alter the transparent or translucent appearance of the present composition. Water soluble plant extracts are preferred. Useful plant extracts herein include clove (choji) extract, coix (yokuinin) extract, witch hazel (hamamerisu) extract, and mixtures thereof.

UV protecting agents generally prevent excessive scaling and texture changes of the stratum corneum by exposure of ultraviolet light and may be added to the emulsion of the present invention. Suitable UV protecting agents may be organic or inorganic. Exact amounts will vary depending upon the sunscreen chosen and the desired Sun Protection Factor (SPF).

Whitening agent useful herein refers to active ingredients that not only alter the appearance of the skin, but further improve hyperpigmentation as compared to pre-treatment. Water soluble whitening agents are preferred. Useful whitening agents herein include ascorbic acid compounds, azelaic acid, butyl hydroxy anisole, glycyrrhizinic acid, hydroquinoine, kojic acid, arbutin, mulberry extract, and mixtures thereof. The preferred ascorbic acid compounds are an ascorbic acid salt or derivative thereof, such as the non-toxic alkali metal, 2-o-α-D-glucopyranosyl-L-ascorbic acid, and its metal salts. Exemplary water soluble salt derivatives include, but are not limited to, L-ascorbic acid 2-glucoside, L-ascorbyl phosphate ester salts such as sodium L-ascorbyl phosphate, magnesium L-ascorbyl phosphate, calcium L-ascorbyl phosphate, aluminum L-ascorbyl phosphate.

Anti-oxidants and radical scavengers are especially useful for providing protection against UV radiation which can cause increased scaling or texture changes in the stratum corneum and against other environmental agents which can cause skin damage. Useful anti-oxidants and radical scavengers herein include tocopherol (vitamin E), tocopherol sorbate, tocopherol acetate, other esters of tocopherol, propyl gallate, alkyl esters of uric acid, amines (i.e., N,N-diethylhydroxylamine, amino-guanidine), sulfhydryl compounds (i.e., glutathione), lycine pidolate, arginine pilolate, bioflavonoids, lysine, methionine, proline, superoxide dismutase, silymarin, tea extracts, grape skin/seed extracts, melanin, and rosemary extracts.

Anti-inflammatory agents enhance the skin appearance benefits, by for example, contribution of uniformity and acceptable skin tone and/or color. Preferably, the anti-inflammatory agent includes a steroidal anti-inflammatory agent such as hydrocortisone and a non-steroidal anti-inflammatory agent. The variety of compounds encompassed by this group is well-known to those skilled in the art.

Other Components

Nonlimiting examples of additional components suitable for use in the deodorant composition include: absorbents; abrasives; anticaking agents; binders; biological actives; bulking agents; buffering agents; chelating agents such as EDTA; cosmetic astringents; chemical additives; colorants; cosmetic biocides; deodorants; dyes and pigments; drying agents; denaturants; drug astringents; external analgesics; essential oils; fragrances; opacifying agents; pH buffering agents; perfumes; propellants; sensates; soothing agents; skin healing agents; vitamins such as vitamin B6; other natural extracts; compounds which stimulate collagen production; Saccharomycopsis ferment filtrate, and others.

The deodorant composition may be a solution, suspension, dispersion, emulsion or other liquid or fluid form, so long as water is the continuous phase.

The deodorant composition of the present invention may also be delivered as a liquid via a spray dispenser or a bottle. Preferred is a manually activated spray dispenser to avoid the use of aerosols which may be irritating to sensitive areas of the body. Spray dispensers useful in the present invention are described more fully in U.S. Pat. No. 5,534,165 which is incorporated herein by reference in its entirety.

Wet Wipe

The deodorant composition of the present invention may be provided in the form of a wet wipe. The wet wipes of the present invention comprise a deodorant composition in addition to the water-insoluble substrate described below. The preferred wet wipe comprises one or more layers of a water-insoluble substrate, and a composition comprising from about 1% to about 10% by weight of the composition of a cyclodextrin, from about 0.10% to about 1.0% by weight of the composition of a film forming polymer, and an aqueous carrier. The wet wipe of the present invention comprises the deodorant composition that impregnates, coats or is otherwise in contact with the water-insoluble substrate described below. For example, the deodorant composition is impregnated at the desired weight onto one or both sides of an absorbent substrate which may be formed from any woven or nonwoven fiber, fiber mixture or foam of sufficient wet strength and absorbency to hold an effective amount of the deodorant composition.

Materials suitable for the deodorant wet wipe of the present invention are well known to those skilled in the art. The wet wipes of the present invention comprise a water-insoluble substrate. By “water insoluble”, it is meant that the substrate does not dissolve in or readily break apart upon immersion in water. The water-insoluble substrate is the implement or vehicle for delivering the deodorant lotion composition to skin in need of removing and/or eliminating body odor.

A wide variety of materials may be used as the substrate. The following nonlimiting characteristics are desirable: (i) sufficient wet strength for use, (ii) sufficient abrasivity, (iii) sufficient loft and porosity, (iv) sufficient thickness, and (v) appropriate size.

Nonlimiting examples of suitable insoluble substrates which meet the above criteria include nonwoven substrates, woven substrates, hydroentangled substrates, air entangled substrates, natural sponges, synthetic sponges, polymeric netted meshes, and the like. Preferred embodiments employ nonwoven substrates since they are economical and readily available in a variety of materials. By “nonwoven”, it is meant that the layer is comprised of fibers which are not woven into a fabric but rather are formed into a sheet, mat, or pad layer. The fibers may either be random (i.e., randomly aligned) or they may be carded (i.e., combed to be oriented in primarily one direction). Furthermore, the nonwoven substrate may be composed of a combination of layers of random and carded fibers.

Methods of making woven and nonwoven substrates are not a part of this invention and, being well known in the art, are not described in detail herein. Generally, however, such substrates are made by air-or water-laying processes in which the fibers or filaments are first cut to desired lengths from long strands, passed into a water or air stream, and then deposited onto a screen through which the fiber-laden air or water is passed. The deposited fibers or filaments are then adhesively bonded together, and otherwise treated as desired to form the woven, nonwoven or cellulose substrates.

Thermocarded nonwoven substrates (whether or not resin-containing) are made of polyesters, polyamides, or other thermoplastic fibers which can be spand bonded, i.e., the fibers are spun out onto a flat surface and bonded (melted) together by heat or chemical reactions.

Nonwoven substrates used in the invention herein are generally adhesively bonded fibers or filamentous products having a web or carded fiber structure (when the fiber strength is suitable to allow carding) or comprising fibrous mats in which the fibers or filaments are distributed haphazardly or in random array (i.e., an array of fibers in a carded web where partial orientation of the fibers is frequently present, as well as a completely haphazard distributional orientation), or substantially aligned. The fibers or filaments can be natural (e.g., wool, silk, jute, hemp, cotton, linen, sisal, or ramie) or synthetic (e.g., rayon, cellulose ester, polyvinyl derivatives, polyolethins, polyamides, or polyesters). These nonwoven materials are generally described in Riedel “Nonwoven Bonding Methods and Materials”, Nonwoven World (1987).

The nonwoven, flexible substrates made from synthetic materials useful in the present invention can be obtained from a wide variety of commercial sources. Nonlimiting examples of suitable nonwoven, flexible substrate materials useful herein include KF-28, a V-groove patterned hydroentangled non-woven substrate having a basis weight of about 60 gsm (grams per square meters), comprising about 55% polyester and about 45% rayon (fiber's length: 50mm), available from Kuraflex in Japan.

Alternatively, the water insoluble substrate may be a polymeric mesh sponge as described in U.S. Pat. No. 5,650,384, incorporated by reference herein in its entirety. The polymeric sponge comprises a plurality of plies of an extruded tubular netting mesh prepared from a strong flexible polymer, such as addition polymers of olefin monomers and polyamides of polycarboxylic acids. Although these polymeric sponges are designed to be used in conjunction with a liquid cleanser, these types of sponges may be used as the water insoluble substrate in the present invention.

The substrate may be made into a wide variety of shapes and forms including flat pads, thick pads, thin sheets, ball-shaped implements, irregularly shaped implements, and having sizes ranging from a surface area of about a square inch (about 6.45 cm²) to about hundreds of square inches (about hundreds of 6.45 cm²). The exact size will depend upon the desired use and product characteristics. Especially convenient are square, circular, rectangular, or oval pads having a surface area of from about 1 in² (about 6.45 cm²) to about 144 in² (about 928.8 cm²), preferably from about 10 in² (about 64.5 cm²) to about 120 in² (about 744 cm2), and more preferably from about 30 in² (about 193.5 cm²) to about 80 in² (about 516 cm²), and a thickness of from about 1 mil (=0.001 inch, i.e., about 0.0254 mm) to about 500 mil (about 10.16 mm), preferably from about 5 mil (about 0.1255 mm) to about 250 mil (about 6.125 mm), and more preferably from about 10 mil (about 0.245 mm) to about 100 mil (about 2.45 mm).

The amount of the deodorant composition associated with any individual wet wipe will vary depending upon the desired characteristics of the finished wet wipe product, but should be at least an amount sufficient to result in deposition of at least some of the deodorant composition onto the skin during application. To that desired end, the substrate comprises the deodorant composition such that the deodorant composition preferably is comprised in an amount of from about 100% to about 400%, more preferably from about 150% to about 350%, still preferably from about 200% to about 300% by weight of the substrate.

Method of Use

The deodorant compositions of the present invention may be topically applied directly to the skin. The composition may be delivered by placing the composition into a dispensing means and applying an effective amount via spraying or rubbing the composition onto the desired skin surface; typically the entire body. Preferably the dispensing means is a wipe comprising a water-insoluble substrate as mentioned above. Distribution of the composition of the present invention can also be achieved by using a pre-formed applicator such as a roller, pad, sponge, tissue cotton ball, hand etc.

Alternatively, the user may combine the composition of the present invention with a substance of his/her own choosing. The user may choose a substance such as a commercial paper towel, tissue, sponge, cotton, pad, washcloth, or the like; and pours, from a bottle or other suitable container, a solution of the composition of the present invention over the chosen substance and applies the composition to the desired area of the body. In this manner, the user may use as much or as little of the composition of the present invention as he/she desires, depending upon their intended use and degree of odor control necessity.

### EXAMPLES

The following examples further describe and demonstrate the preferred embodiments within the scope of the present invention. The examples are given solely for the purpose of illustration and are not to be construed as limitations of the present invention since many variations thereof are possible without departing from the spirit and scope of the invention. Ingredients are identified by chemical name, or otherwise defined below.

All ingredients herein are based upon the total weight of the compositions, and all such weight percentages as they pertain to listed ingredients are based on the active level and, therefore, do not include carriers or by-products that may be included in commercially available materials.

The composition of the present invention may be prepared by techniques commonly known in the art. A person skilled in the art may readily understand how to prepare the composition to make it homogeneous based on the formulation shown below. Reference may be made for such techniques to Remington's Pharmaceutical Science, 18^{th} Edition, 1990.

The following Examples 1-5 are non-limiting examples of embodiments for lotion composition of the present invention.

| | Ex. 1 | Ex. 2 | Ex. 3 | Ex. 4 | Ex. 5 |
|---|---|---|---|---|---|
| Ingredients | Wt. % | Wt. % | Wt. % | Wt. % | Wt. % |
| Alpha-cyclodextrin *1 | | 0.5 | 2.5 | 2.5 | |
| Beta-cyclodextrin *2 | | 2.0 | | | 3.0 |
| 2-Hydroxypropyl beta- | 3.0 | 2.5 | | 3.5 | |
| cyclodextrin *3 | | | | | |
| Methylated beta-cyclodextrin *4 | | | 5.5 | | 3.0 |
| Polyquaternium-39 *5 | 0.4 | 0.2 | 0.3 | 0.5 | 0.5 |
| Dipropylene glycol *6 | 7.0 | | 7.0 | 7.0 | |
| 1,3-butyleneglycol *7 | 3.0 | | 3.0 | | 3.0 |
| Glycerol *8 | 4.0 | 4.0 | | 4.0 | |
| Polyethylene glycol 1540 *9 | 0.5 | | 0.5 | 0.5 | |
| Polysorbate 20 *10 | 0.2 | | 0.20 | | 0.2 |
| Methylparaben *11 | 0.11 | 0.11 | | 0.11 | 0.11 |
| Benzyl alcohol *12 | 0.10 | | 0.10 | | 0.10 |
| Isopropyl palmitate | | 0.05 | 0.05 | 0.1 | |
| Dimethicone *13 | | | 2.00 | | 2.00 |
| Niacinamide *14 | | 0.5 | | 0.5 | 1.0 |
| Panthenol *15 | | | 1.0 | 1.0 | 1.0 |
| Clove extract *16 | | 0.05 | | 1.0 | |
| Saccharomycopsis Ferment | | 0.1 | 0.01 | | 0.01 |
| Filtrate *17 | | | | | |
| Mulberry extract | | | 0.1 | | 0.05 |
| L-ascorbic acid 2-glucoside | | 0.1 | | 0.05 | |
| Purified water | balance | balance | balance | balance | balance |
| Total | 100 | 100 | 100 | 100 | 100 |

*1 Alpha-cyclodextrin: Cavamax W6 available from Wacker
*2 Beta-cyclodextrin: Cavamax W7 available from Wacker
*3 2-Hydroxypropyl beta-cyclodextrin: Cavasol W7HP available from Wacker
*4 Methylated beta-cyclodextrin: Cavasol W7M available from Wacker
*5 Polyquaternium-39: Merquat Plus 3330 available from Cargon
*6 Dipropylene glycol: Dipropylene glycol available from Daicel K. K.
*7 1,3-Butylene Glycol: 1,3-Butylene Glycol available from Daidel K. K.
*8 Glycerol: Glycerl available from Nippon Oil & Fat Company Ltd.
*9 Polyethylene glycol 1540: Polyethylene glycol 1540 available from Nippon Oil & Fat Co.
*10 Polysorbate 20: Tween 20 available from ICI Surfactant
*11 Methylparaben: Methylparaben available from Ueno Pharmaceutical Co.
*12 Benzyl alcohol: Benzyl alcohol available from Takasago
*13 Dimethicone: Dow Corning 200 Fluid available from Dow Corning
*14 Niacinamide: Niacinamide available from Roche
*15 Panthenol: DL-Panthenol available from Roche
*16 Clove extract: Clove extract available from Iwase
*17 Saccharomycopsis Ferment Filtrate: SK-II Pitera available from Kashiwayama

Example 6 is a non-limiting example of a wet-wipe composition of the present invention

### Example 6

KF-28, a V-groove patterned hydroentangled non-woven substrate having a basis weight of about 60 gsm (grams per square meters), comprising about 55% polyester and about 45% rayon (fiber's length: 50 mm), available from Kuraflex in Japan was used for the present invention.

The substrate contained the composition of the present invention about 250% by weight of the substrate. The composition comprised 3.0 wt % of hydroxypropyl beta-cyclodextrin, 0.4 wt % of polyquaternium-39, 7.0 wt % of dipropylene glycol, 3.0 wt % of 1,3-butylene glycol, 4.0 wt % of glycerol, 0.5 wt % of polyethylene glycol 1540, 0.2 wt % of polysorbate 20, 0.11 wt % of methyl paraben, 0.10 wt % of benzyl alcohol, and 78.69 wt % of purified water.

These embodiments represented by the previous examples provide improved perspiration malodor control with less resulting in skin irritation and provide long lasting deodorant benefit after the compositions have been topically applied to the skin.

What is claimed is:

1. A composition comprising:
a cyclodextrin;
a film forming polymer, and
an aqueous carrier.

2. The composition according to claim 1 further comprising a component selected from the group consisting of emollients, moisturizers, skin protectants, and mixtures thereof.

3. The composition according to claim 1 further comprising an antimicrobial agent.

4. The composition according to claim 1 comprising the cyclodextrin at concentration from about 1% to about 10% by weight of the composition.

5. The composition according to claim 1 comprising the film forming polymer at concentration from about 0.10% to about 0.5% by weight of the composition.

6. The composition according to claim 1, wherein the cyclodextrin is selected from the group consisting of alpha-cyclodextrin, beta-cyclodextrin, methyl-alpha-cyclodextrin, methyl-beta-cyclodextrin, hydroxypropyl-alpha-cyclodextrin, hydroxypropyl-beta-cyclodextrin, and mixtures thereof.

7. The composition according to claim 6, wherein the cyclodextrin is beta-cyclodextrin.

8. The composition according to claim 1, wherein the film-forming polymer is a water soluble polymer having cationic functionalities.

9. The composition according to claim 1, wherein the film-forming polymer is polyquaternium-39.

10. A wet wipe comprising:
one or more layers of a water-insoluble substrate; and
a composition comprising:
a cyclodextrin at concentration from about 1.0% to about 10.0% by weight of the composition;
a film forming polymer at concentration from about 0.10% to about 0.5% by weight of the composition, and an aqueous carrier.

11. The wet wipe according to claim 10 wherein the deodorant composition is comprised in an amount of from about 100% to about 400% by weight of the substrate.
